SEQUENCE LISTING (28 sequences)

SEQ ID NO: 1 - protein, 8 aa, Artificial
  Linker
ERKGGGSG

SEQ ID NO: 2 - protein, 9 aa, Artificial
  Linker
ERKGGGSGS

SEQ ID NO: 3 - protein, 16 aa, Artificial
  Linker
AKTTPKLEEGEFSEAR

SEQ ID NO: 4 - protein, 17 aa, Artificial
  Linker
AKTTPKLEEGEFSEARV

SEQ ID NO: 5 - protein, 9 aa, Artificial
  Linker
AKTTPKLGG

SEQ ID NO: 6 - protein, 10 aa, Artificial
  Linker
SAKTTPKLGG

SEQ ID NO: 7 - protein, 17 aa, Artificial
  Linker
AKTTPKLEEGEFSEARV

SEQ ID NO: 8 - protein, 6 aa, Artificial
  Linker
SAKTTP

SEQ ID NO: 9 - protein, 10 aa, Artificial
  Linker
SAKTTPKLGG

SEQ ID NO: 10 - protein, 6 aa, Artificial
  Linker
RADAAP

SEQ ID NO: 11 - protein, 9 aa, Artificial
  Linker
RADAAPTVS

SEQ ID NO: 12 - protein, 12 aa, Artificial
  Linker
RADAAAAGGPGS

SEQ ID NO: 13 - protein, 27 aa, Artificial
  Linker
RADAAAAGGGGSGGGGSGGGGSGGGGS

SEQ ID NO: 14 - protein, 6 aa, Artificial
  Linker
SAKTTP

SEQ ID NO: 15 - protein, 10 aa, Artificial
  Linker
SAKTTPKLGG

SEQ ID NO: 16 - protein, 18 aa, Artificial
  Linker
SAKTTPKLEEGEFSEARV

SEQ ID NO: 17 - protein, 5 aa, Artificial
  Linker
ADAAP

SEQ ID NO: 18 - protein, 12 aa, Artificial
  Linker
ADAAPTVSIFPP

SEQ ID NO: 19 - protein, 5 aa, Artificial
  Linker
TVAAP

SEQ ID NO: 20 - protein, 12 aa, Artificial
  Linker
TVAAPSVFIFPP

SEQ ID NO: 21 - protein, 6 aa, Artificial
  Linker
QPKAAP

SEQ ID NO: 22 - protein, 13 aa, Artificial
  Linker
QPKAAPSVTLFPP

SEQ ID NO: 23 - protein, 6 aa, Artificial
  Linker
AKTTPP

SEQ ID NO: 24 - protein, 13 aa, Artificial
  Linker
AKTTPPSVTPLAP

SEQ ID NO: 25 - protein, 6 aa, Artificial
  Linker
AKTTAP

SEQ ID NO: 26 - protein, 13 aa, Artificial
  Linker
AKTTAPSVYPLAP

SEQ ID NO: 27 - protein, 6 aa, Artificial
  Linker
ASTKGP

SEQ ID NO: 28 - protein, 13 aa, Artificial
  Linker
ASTKGPSVFPLAP

(19) United States
(12) Patent Application Publication
(10) Pub. No.: US 2013/0115215 A1
(43) Pub. Date: May 9, 2013

(54) DOMAIN INSERTION IMMUNOGLOBULIN

(71) Applicant: Hongxing Zhou, Bellevue, WA (US)

(72) Inventor: Hongxing Zhou, Bellevue, WA (US)

(21) Appl. No.: 13/809,577

(22) PCT Filed: Jul. 14, 2011
(86) PCT No.: PCT/US2011/044020

§ 371 Date: Jan. 10, 2013

Related U.S. Application Data
(60) Provisional application No. 61/364,315, filed on Jul. 14, 2010.

(51) Int. Cl. A61K 39/395; C12P 21/04; C07H 21/04; C07K 14/705; C07K 16/30

(52) U.S. Cl. 424/136.1; 530/387.3; 536/23.53; 435/328; 435/69.6

(57) ABSTRACT

Described herein is an antibody format, which is amenable to bispecific antibody creation. This format is referred to herein as “Domain Insertion Immunoglobulin G” or “(Di-IgG)”. The Di-IgG molecules are capable of specifically binding two different antigens simultaneously, show high level recombinant expression, and are sufficiently aggregation-free to be amenable to commercial production. Further described herein are, Di-IgG-encoding nucleic acids and vectors, host cells for making Di-IgGs, Di-IgG pharmaceutical compositions, and methods of treatment.

### CROSS-REFERENCE TO RELATED APPLICATIONS

This application claims the benefit of U.S. Provisional Application No. 61/364,315, filed Jul. 14, 2010, which is hereby incorporated by reference.

### BACKGROUND OF THE INVENTION

Bispecific antibodies hold great promise as next-generation human biotherapeutics. Because a bispecific antibody offers many unique advantages, great efforts have been invested in the field of engineering these types of molecules. Unfortunately, the investments have resulted in limited success. Among the most common issues is the difficulty in scaling up to large-scale production of these types of molecules. Bispecific antibodies have been notoriously low in expression yield and have a high tendency to aggregate, resulting in heterogeneous product. These production issues have made the commercialization of bispecific antibody difficult if not essentially impossible.

US20090215992 proposes a dual-variable domain antibody. Similar to a diabody, the dual-variable domain antibody of US20090215992 comprises two variable domains connected together, preferably by a short linker. Specifically, the additional variable domain is connected to the N-terminus of both the heavy and the light chains of an antibody.

### SUMMARY OF THE INVENTION

Described herein is a novel antibody format, which is amenable to bispecific antibody creation. This format is referred to herein as “Domain Insertion Immunoglobulin G” or “(Di-IgG)”. However, as will be apparent to one of skill in the art from the description, the invention is not limited to use with IgG antibodies but may be applied to the other antibody isotypes. Moreover, the invention is not limited to intact antibodies but also includes antibody fragments.

Rather than connecting the additional variable domain to the N-terminus of each chain, the Di-IgG molecule is created by inserting a variable domain into the heavy chain in proximity to the CH1 domain, preferably between the CH1 domain and the antibody hinge region, and by fusing a variable domain to the C-terminus of the light chain of an antibody. As demonstrated in the Examples, Di-IgG molecules are capable of specifically binding two different antigens simultaneously. Moreover, they show high level recombinant expression and are sufficiently aggregation-free to be amenable to commercial production. Thus, the Di-IgG platform is a novel and robust platform to make bi-specific antibodies useful as human therapeutics.

A Di-IgG molecule may be used in a number of ways, including, but not limited to:

1) A Di-IgG molecule can bind two different molecules simultaneously. As a therapeutic molecule, this is advantageous because oftentimes a biological pathway of a human disease is modulated by two molecules or two different pathological pathways lead to the same human disease. Under both circumstances, an effective treatment will require only a single drug made of a Di-IgG molecule while requiring two drugs if each are monospecific antibodies. For example, II-4 and IL-13 are two distinct cytokines that are both implicated in the development of allergic asthma. Because these two cytokines do not share common domains or obvious epitope motifs in common for binding antibodies, it would require two monospecific blocking antibodies to inhibit their pathological activities whereas a single Di-IgG molecule can potentially achieve the same goal;

2) A Di-IgG molecule also can be used to retarget a specific molecule to a predetermined location. For example, a Di-IgG molecule specific to IL-2 and a tumor specific antigen can be used to selectively enrich IL-2 in the vicinity of a tumor cell;

3) A Di-IgG molecule can be used to retarget one specific type of cell to another type of cell. For example, a Di-IgG molecule can be engineered to bind to T cells and tumor cells. This Di-IgG molecule can be used to kill tumor cells specifically and potently by retargeting the T-cell cytotoxicity; and

4) A Di-IgG molecule can be used to bridge two different receptor molecules in a way similar to what a heterodimeric receptor cytokine does to activate the receptor, thereby serving as a receptor agonist. A Di-IgG molecule recognizing IL-2 receptor β and γ chains can potentially be used to mimic the function of IL-2.

In a first aspect of the invention, an antigen binding protein comprises a first polypeptide chain and a second polypeptide chain, wherein said first polypeptide chain comprises VH1-CH1-X-VH2, wherein:

VH1 is a first heavy chain variable domain;

CH1 is a heavy chain constant domain;

X is an optional linker;

VH2 is a second heavy chain variable domain; and

wherein said second polypeptide chain comprises VL1-CL-X-VL2, wherein:

VL1 is a first light chain variable domain;

CL is a light chain constant domain;

X is an optional linker;

VL2 is a second light chain variable domain.

The first polypeptide chain may further comprise a native or variant Fc. The Fc may be selected from the group consisting of an Fc region from an IgA, IgD, IgE, IgG1, IgG2, IgG3, IgG4, and IgM. CL may be a kappa or lambda light chain constant domain.

Each optional linker may comprise anywhere from 3 to 50 amino acids. Preferably, the linker comprises a plurality of glycines, e.g., GluArgLysGlyGlyGlySerGly.

In certain embodiments of the first aspect, the antigen binding protein comprises two first polypeptide chains and two second polypeptide chains.

In some embodiments of the first aspect, VH1 and VL1 are obtained from a parent antibody or from a parent antigen binding portion of an antibody and/or VH2 and VL2 are obtained from a parent antibody or from a parent antigen binding portion of an antibody. In other embodiments, the VH1 and VL1 are obtained from a different parent antibody or from a different parent antigen binding portion of an antibody and/or the VH2 and VL2 are obtained from a different parent antibody or from a different parent antigen binding portion of an antibody. In certain embodiments, the VH1, VL1, VH2, and VL2 are obtained from the same parent antibody or from the same parent antigen binding portion of an antibody.

In preferred embodiment of the first aspect, the VH1 and VL1 specifically bind a first antigen and the VH2 and VL2 specifically bind a second antigen. The first antigen and second antigen may be the same or different antigens. When the same antigen, the VH1 and VL1 may specifically bind a first epitope on the antigen and the VH2 and VL2 may specifically bind a second epitope on the antigen.

In a second aspect of the invention, a nucleic acid encodes a first polypeptide chain comprising VH1-CH1-X-VH2, wherein:

VH1 is a first heavy chain variable domain;

CH1 is a heavy chain constant domain;

X is an optional linker;

VH2 is a second heavy chain variable domain.

In a third aspect of the invention, a nucleic acid encodes a second polypeptide chain comprising VL1-CL-X-VL2, wherein:

VL1 is a first light chain variable domain;

CL is a light chain constant domain;

X is an optional linker;

VL2 is a second light chain variable domain.

In a forth aspect of the invention, a vector comprises the nucleic aspect of the second aspect and/or the nucleic acid of the third aspect.

In a fifth aspect, a host cell comprises the nucleic acid of the second aspect, the nucleic acid of the third aspect, and/or the vector of the forth aspect.

In a sixth aspect, an antigen binding protein is made by a method comprising:

a) culturing a host cell of the fifth aspect in a culture medium under conditions such that the first polypeptide and second polypeptide are expressed to create a culture; and

b) isolating the antigen binding protein from the culture. The antigen binding protein may be isolated from the cells or the culture medium.

In a seventh aspect, a pharmaceutical composition comprises an antigen binding protein the first aspect and a pharmaceutically acceptable excipient.

### BRIEF DESCRIPTION OF THE DRAWINGS

FIG. 1 Structural comparison of embodiments of the invention to a standard antibody (IgG) and the dual-variable domain antibody of US20090215992 (DVD-IgG).

FIG. 2A-C. FIG. 2A demonstrates that a huIL-12/muCD40 Di-IgG is capable of binding human IL-12 similarly to the parent antibody. FIG. 2B demonstrates that a huIL-12/muCD40 Di-IgG is capable of binding murine CD40 similarly to the parent antibody. FIG. 2C demonstrates that a huIL-12/muCD40 Di-IgG is capable of binding human IL-12 and murine CD40 simultaneously while a parent antibody does not.

### DETAILED DESCRIPTION OF PREFERRED EMBODIMENTS OF THE INVENTION

The section headings used herein are for organizational purposes only and are not to be construed as limiting the subject matter described. All references cited within the body of this specification are expressly incorporated by reference in their entirety.

Standard techniques may be used for recombinant DNA, oligonucleotide synthesis, tissue culture and transformation, protein purification, etc. Enzymatic reactions and purification techniques may be performed according to the manufacturer's specifications or as commonly accomplished in the art or as described herein. The following procedures and techniques may be generally performed according to conventional methods well known in the art and as described in various general and more specific references that are cited and discussed throughout the specification. See, e.g., Sambrook et al., 2001, Molecular Cloning: A Laboratory Manuel, 3^{rd} ed., Cold Spring Harbor Laboratory Press, cold Spring Harbor, N.Y., which is incorporated herein by reference for any purpose. Unless specific definitions are provided, the nomenclature used in connection with, and the laboratory procedures and techniques of, analytic chemistry, organic chemistry, and medicinal and pharmaceutical chemistry described herein are those well known and commonly used in the art. Standard techniques may be used for chemical synthesis, chemical analyses, pharmaceutical preparation, formulation, and delivery and treatment of patients.

Described herein are multivalent and multispecific antigen binding proteins, including the components of the antigen binding proteins which include polypeptides comprising two or more antibody variable regions. The antigen binding proteins of the invention may be formulated into a pharmaceutical composition and used to treat one or more diseases. Further embodiments include nucleic acids encoding one or more polypeptides that make up the antigen binding protein, expression vectors that include such nucleic acids, and host cells for making such antigen binding proteins.

The multivalent and multispecific antigen binding proteins specifically bind to one or more antigens. “Specifically binds” as used herein means that the antigen binding protein preferentially binds the antigen over other proteins. In some embodiments “specifically binds” means the antigen binding protein has a higher affinity for the antigen than for other proteins. Antigen binding proteins that specifically bind an antigen may have a binding affinity for the antigen of less than or equal to 1×10⁻⁷ M, less than or equal to 2×10⁻⁷ M, less than or equal to 3×10⁻⁷ M, less than or equal to 4×10⁻⁷ M, less than or equal to 5×10⁻⁷ M, less than or equal to 6×10⁻⁷ M, less than or equal to 7×10⁻⁷ M, less than or equal to 8×10⁻⁷ M, less than or equal to 9×10⁻⁷ M, less than or equal to 1×10⁻⁸ M, less than or equal to 2×10⁻⁸ M, less than or equal to 3×10⁻⁸ M, less than or equal to 4×10⁻⁸ M, less than or equal to 5×10⁻⁸ M, less than or equal to 6×10⁻⁸ M, less than or equal to 7×10⁻⁸ M, less than or equal to 8×10⁻⁸ M, less than or equal to 9×10⁻⁸ M, less than or equal to 1×10⁻⁹ M, less than or equal to 2 x 10⁻⁹ M, less than or equal to 3×10⁻⁹ M, less than or equal to 4×10⁻⁹ M, less than or equal to 5×10⁻⁹ M, less than or equal to 6×10⁻⁹ M, less than or equal to 7×10⁻⁹ M, less than or equal to 8×10⁻⁹ M, less than or equal to 9×10⁻⁹ M, less than or equal to 1×10⁻¹⁰ M, less than or equal to 2×10⁻¹⁰ M, less than or equal to 3×10⁻¹⁰ M, less than or equal to 4×10⁻¹⁰ M, less than or equal to 5×10⁻¹⁰ M, less than or equal to 6×10⁻¹⁰ M, less than or equal to 7×10⁻¹⁰ M, less than or equal to 8×10⁻¹⁰ M, less than or equal to 9×10⁻¹⁰ M, less than or equal to 1×10⁻¹¹ M, less than or equal to 2×10⁻¹¹ M, less than or equal to 3×10⁻¹¹ M, less than or equal to 4×10⁻¹¹ M, less than or equal to 5×10⁻¹¹ M, less than or equal to 6×10⁻¹¹ M, less than or equal to 7×10⁻¹¹ M, less than or equal to 8×10⁻¹¹ M, less than or equal to 9×10⁻¹¹ M, less than or equal to 1×10⁻¹² M, less than or equal to 2×10⁻¹² M, less than or equal to 3×10⁻¹² M, less than or equal to 4×10⁻¹² M, less than or equal to 5×10⁻¹² M, less than or equal to 6×10⁻¹² M, less than or equal to 7×10⁻¹² M, less than or equal to 8×10⁻¹² M, or less than or equal to 9×10⁻¹² M.

An antigen binding protein of the invention may be comprised of two or more polypeptides. In certain embodiments, the antigen binding protein comprises a first polypeptide chain having the general formula VH1-CH1-X-VH2, wherein VH1 is a heavy chain variable domain, CH1 is a heavy chain constant domain 1, X is an optional linker, and VH2 is a second heavy chain variable domain. The antigen binding protein will often include a second polypeptide having the general formula VL1-CL1-X-VL2, wherein VL1 is a light chain variable domain, CL1 is a light chain constant domain, X is an optional linker, and VL2 is a second light chain variable domain. In preferred embodiments, the VH1 and VL1 associate to specifically bind to an antigen and the VH2 and VL2 associate to specifically bind an antigen. In other preferred embodiments the first polypeptide chain comprises an Fc portion of an antibody C-terminal of the VH2.

The variable regions of the heavy and light chains typically exhibit the same general structure of relatively conserved framework regions (FR) joined by three hypervariable regions, i.e., the complementarity determining regions or CDRs. The CDRs are primarily responsible for antigen recognition and binding. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3, and FR4. The assignment of amino acids to each domain is in accordance with the definitions of Kabat.

CDRs constitute the major surface contact points for antigen binding. The CDR3 or the light chain and, particularly, CDR3 of the heavy chain may constitute the most important determinants in antigen binding within the light and heavy chain variable regions. In some antibodies, the heavy chain CDR3 appears to constitute the major area of contact between the antigen and the antibody. In vitro selection schemes in which CDR3 alone is varied can be used to vary the binding properties of an antibody or determine which residues contribute to the binding of an antigen.

The terms “Kabat numbering”, “Kabat definitions and “Kabat labeling” may be used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, IL2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):73245 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

A “linker” may comprise two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acid residues joined by peptide bonds. In preferred embodiments, a linker is used to link one or more antigen binding portions and said linker is heterologous to the native antibody sequence occurring in the portion of the antigen binding protein. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). Preferred linkers include, but are not limited to, ERKGGGSG, ERKGGGSGS, AKTTPKLEEGEFSEAR; AKTTPKLEEGEFSEARV; AKTTPKLGG; SAKTTPKLGG; AKTTPKLEEGEFSEARV; SAKTTP; SAKTTPKLGG; RADAAP; RADAAPTVS; RADAAAAGGPGS; RADAAAA(G₄S)₄; SAKTTP; SAKTTPKLGG; SAKTTPKLEEGEFSEARV; ADAAP; ADAAPTVSIFPP; TVAAP; TVAAPSVFIFPP; QPKAAP; QPKAAPSVTLFPP; AKTTPP; AKTTPPSVTPLAP; AKTTAP; AKTTAPSVYPLAP; ASTKGP; and ASTKGPSVFPLAP.

It is contemplated that essentially any antibody variable domain or antigen binding portion thereof (e.g., CDR3) may be incorporated into the Di-IgG format. Exemplary antibody variable domains (and the antigen to which they specifically bind) include, but are not limited to, those described in U.S. Pat. No. 7,947,809 and US20090041784 (glucagon receptor), U.S. Pat. No. 7,939,070, U.S. Pat. No. 7,833,527, U.S. Pat. No. 7,767,206, and U.S. Pat. No. 7,786,284 (IL-17 receptor A), U.S. Pat. No. 7,872,106 and U.S. Pat. No. 7,592,429 (Sclerostin), U.S. Pat. No. 7,871,611, U.S. Pat. No. 7,815,907, U.S. Pat. No. 7,037,498, U.S. Pat. No. 7,700,742, and US20100255538 (IGF-1 receptor), U.S. Pat. No. 7,868,140 (B7RP1), U.S. Pat. No. 7,807,159 and US20110091455 (myostatin), U.S. Pat. No. 7,736,644, U.S. Pat. No. 7,628,986, U.S. Pat. No. 7,524,496, and US20100111979 (deletion mutants of epidermal growth factor receptor), U.S. Pat. No. 7,728,110 (SARS coronavirus), U.S. Pat. No. 7,718,776 and US20100209435 (OPGL), U.S. Pat. No. 7,658,924 and U.S. Pat. No. 7,521,053 (Angiopoietin-2), U.S. Pat. No. 7,601,818, U.S. Pat. No. 7,795,413, US20090155274, and US20110040076 (NGF), U.S. Pat. No. 7,579,186 (TGF-β type II receptor), U.S. Pat. No. 7,541,438 (connective tissue growth factor), U.S. Pat. No. 7,438,910 (IL1-R1), U.S. Pat. No. 7,423,128 (properdin), U.S. Pat. No. 7,411,057, U.S. Pat. No. 7,824,679, U.S. Pat. No. 7,109,003, U.S. Pat. No. 6,682,736, U.S. Pat. No. 7,132,281, and U.S. Pat. No. 7,807,797 (CTLA-4), U.S. Pat. No. 7,084,257, U.S. Pat. No. 7,790,859, U.S. Pat. No. 7,335,743, U.S. Pat. No. 7,084,257, and US20110045537 (interferon-gamma), U.S. Pat. No. 7,932,372 (MAdCAM), U.S. Pat. No. 7,906,625, US20080292639, and US20110044986 (amyloid), U.S. Pat. No. 7,815,907 and U.S. Pat. No. 7,700,742 (insulin-like growth factor I), U.S. Pat. No. 7,566,772 and U.S. Pat. No. 7,964,193 (interleukin-1β), U.S. Pat. No. 7,563,442, U.S. Pat. No. 7,288,251, U.S. Pat. No. 7,338,660, U.S. Pat. No. 7,626,012, U.S. Pat. No. 7,618,633, and US20100098694 (CD40), U.S. Pat. No. 7,498,420 (c-Met), U.S. Pat. No. 7,326,414, U.S. Pat. No. 7,592,430, and U.S. Pat. No. 7,728,113 (M-CSF), U.S. Pat. No. 6,924,360, U.S. Pat. No. 7,067,131, and U.S. Pat. No. 7,090,844 (MUC18), U.S. Pat. No. 6,235,883, U.S. Pat. No. 7,807,798, and US20100305307 (epidermal growth factor receptor), U.S. Pat. No. 6,716,587, U.S. Pat. No. 7,872,113, U.S. Pat. No. 7,465,450, U.S. Pat. No. 7,186,809, U.S. Pat. No. 7,317,090, and U.S. Pat. No. 7,638,606 (interleukin-4 receptor), US20110135657 (BETA-KLOTHO), U.S. Pat. No. 7,887,799 and U.S. Pat. No. 7,879,323 (fibroblast growth factor-like polypeptides), U.S. Pat. No. 7,867,494 (IgE), US20100254975 (ALPHA-4 BETA-7), US20100197005 and U.S. Pat. No. 7,537,762 (ACTIVIN RECEPTOR-LIKE KINASE-1), U.S. Pat. No. 7,585,500 and US20100047253 (IL-13), US20090263383 and U.S. Pat. No. 7,449,555 (CD148), US20090234106 (ACTIVIN A), US20090226447 (angiopoietin-1 and angiopoietin-2), US20090191212 (Angiopoietin-2), US20090155164 (C-FMS), U.S. Pat. No. 7,537,762 (activin receptor-like kinase-1), U.S. Pat. No. 7,371,381 (galanin), US20070196376 (INSULIN-LIKE GROWTH FACTORS), U.S. Pat. No. 7,267,960 and U.S. Pat. No. 7,741,115 (LDCAM), U.S. Pat. No. 7,265,212 (CD45RB), U.S. Pat. No. 7,709,611, US20060127393 and US20100040619 (Dkk-1), U.S. Pat. No. 7,807,795, US20030103978 and U.S. Pat. No. 7,923,008 (osteoprotegerin), US20090208489 (OV064), US20080286284 (PSMA), U.S. Pat. No. 7,888,482, US20110165171, and US20110059063 (PAR2), US20110150888 (HEPCIDIN), U.S. Pat. No. 7,939,640 (B7L-1), U.S. Pat. No. 7,915,391 (c-Kit), U.S. Pat. No. 7,807,796, U.S. Pat. No. 7,193,058, and U.S. Pat. No. 7,427,669 (ULBP), U.S. Pat. No. 7,786,271, U.S. Pat. No. 7,304,144, and US20090238823 (TSLP), U.S. Pat. No. 7,767,793 (SIGIRR), U.S. Pat. No. 7,705,130 (HER-3), U.S. Pat. No. 7,704,501 (ataxin-1-like polypeptide), U.S. Pat. No. 7,695,948 and U.S. Pat. No. 7,199,224 (TNF-α converting enzyme), US20090234106 (ACTIVIN A), US20090214559 and U.S. Pat. No. 7,438,910 (IL1-R1), U.S. Pat. No. 7,579,186 (TGF-β type II receptor), U.S. Pat. No. 7,569,387 (TNF receptor-like molecules), U.S. Pat. No. 7,541,438, (connective tissue growth factor), U.S. Pat. No. 7,521,048 (TRAIL receptor-2), U.S. Pat. No. 6,319,499, U.S. Pat. No. 7,081,523, and US20080182976 (erythropoietin receptor), US20080166352 and U.S. Pat. No. 7,435,796 (B7RP1), U.S. Pat. No. 7,423,128 (properdin), U.S. Pat. No. 7,422,742 and U.S. Pat. No. 7,141,653 (interleukin-5), U.S. Pat. No. 6,740,522 and U.S. Pat. No. 7,411,050 (RANKL), U.S. Pat. No. 7,378,091 (carbonic anhydrase IX (CA IX) tumor antigen), U.S. Pat. No. 7,318,925and U.S. Pat. No. 7,288,253 (parathyroid hormone), U.S. Pat. No. 7,285,269 (TNF), U.S. Pat. No. 6,692,740 and U.S. Pat. No. 7,270,817 (ACPL), U.S. Pat. No. 7,202,343 (monocyte chemo-attractant protein-1), U.S. Pat. No. 7,144,731 (SCF), U.S. Pat. No. 6,355,779 and U.S. Pat. No. 7,138,500 (4-1BB), U.S. Pat. No. 7,135,174 (PDGFD), U.S. Pat. No. 6,630,143 and U.S. Pat. No. 7,045,128 (Flt-3 ligand), U.S. Pat. No. 6,849,450 (metalloproteinase inhibitor), U.S. Pat. No. 6,596,852 (LERK-5), U.S. Pat. No. 6,232,447 (LERK-6), U.S. Pat. No. 6,500,429 (brain-derived neurotrophic factor), U.S. Pat. No. 6,184,359 (epithelium-derived T-cell factor), U.S. Pat. No. 6,143,874 (neurotrophic factor NNT-1), US20110027287 (PROPROTEIN CONVERTASE SUBTILISIN KEXIN TYPE 9 (PCSK9)), US20110014201 (IL-18 RECEPTOR), and US20090155164 (C-FMS). The above patents and published patent applications are incorporated herein by reference in their entirety for purposes of their disclosure of variable domain polypeptides, variable domain encoding nucleic acids, host cells, vectors, methods of making polypeptides encoding said variable domains, pharmaceutical compositions, and methods of treating diseases associated with the respective target of the variable domain-containing antigen binding protein or antibody.

In preferred embodiments, the Di-IgG molecule is created by inserting a variable domain into or proximally C-terminal to the CH1 domain of an antibody heavy chain. In determining the location in which the variable domain is inserted, care should be taken as to not disrupt the structure or stability of the parental antibody significantly.

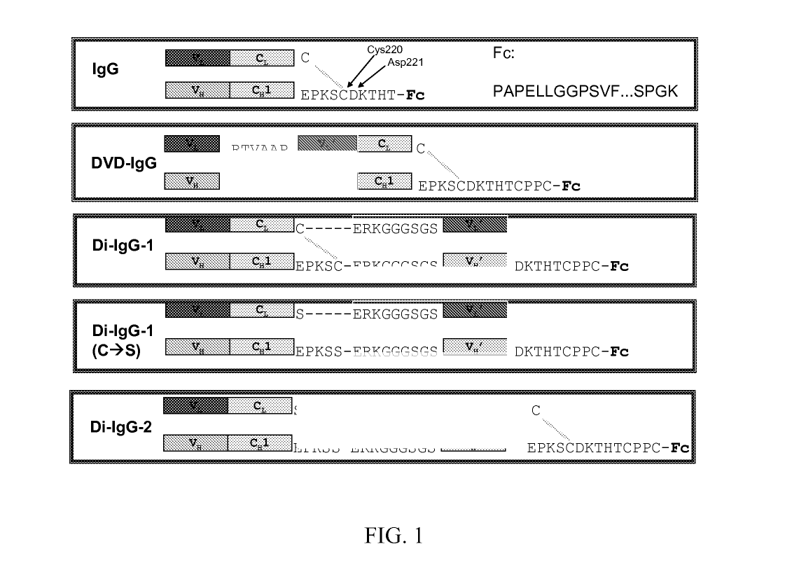

In some embodiments the variable domain along with a peptide linker at its N-terminus is inserted into the heavy chain C-terminal of the cysteine residue that forms a di-sulfide bond with the light chain. (FIG. 1; Di-IgG-1) In certain embodiments, the cysteine residue is mutated such to prevent the formation of a di-sulfide bond with the light chain. (FIG. 1; Di IgG-1 (C→S) In other embodiments, the variable domain is inserted into the heavy chain N-terminal of the cysteine residue that forms a di-sulfide bond with the light chain. (FIG. 1; Di-IgG-2).

In preferred embodiments, the Di-IgG further comprises a light chain having a second variable domain following the CL domain. In some embodiments, the second variable domain is preceded by a linker. (FIG. 1).

Embodiments of the invention include mono-, dual-, tri-,and quad-specific antibodies, depending on the combination of variable domains used to create the Di-IgG. For example, one arm of the antibody may contain a variable domain for binding target A and a variable domain for binding target B, while the other arm contains a variable domain for binding target C, thereby creating a tri-specific antibody.

WO2009089004 (incorporated herein by reference in its entirety) describes compositions and methods for engineering the CH3 domain interface to decrease homodimerization and increase heterodimerization between two CH3-containing molecules. The methods relied on changing the electrostatic charge of key residues within the interface. Similar type changes could be made in the CH1 domain and the CL domain. Using such technology, one could create tri- or even quad-valent/specific antigen binding proteins in an efficient manner. Thus, in some embodiments an antigen binding protein may be capable of binding three or even four different antigens or three or four different epitopes on a single antigen.

Antibodies having multiple variable domains have been described in the art. U.S. Patent Appl. Pub. 2009/0215992. Though the present invention differs significantly from the invention described in 2009/0215992, much of the nomenclature, components, and use of such antibodies, including various combinations of targets, methods of treating, pharmaceutical formulations, linkers, administration routes, etc, are common between the two inventions. Patent Appl. Pub. 2009/0215992 is incorporated herein by reference in its entirety

### Pharmaceutical Compositions

In some embodiments, the invention provides a pharmaceutical composition comprising a therapeutically effective amount of one or a plurality of the antigen binding proteins of the invention together with a pharmaceutically effective diluents, carrier, solubilizer, emulsifier, preservative, and/or adjuvant. Pharmaceutical compositions of the invention include, but are not limited to, liquid, frozen, and lyophilized compositions.

Preferably, formulation materials are nontoxic to recipients at the dosages and concentrations employed. In specific embodiments, pharmaceutical compositions comprising a therapeutically effective amount of an antigen binding protein, e.g., Di-IgG, are provided.

In certain embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine, proline, or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

In certain embodiments, the optimal pharmaceutical composition will be determined by one skilled in the art depending upon, for example, the intended route of administration, delivery format and desired dosage. See, for example, REMINGTON'S PHARMACEUTICAL SCIENCES, supra. In certain embodiments, such compositions may influence the physical state, stability, rate of in vivo release and rate of in vivo clearance of the antigen binding proteins of the invention. In certain embodiments, the primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and may further include sorbitol or a suitable substitute therefor. In certain embodiments of the invention, a antigen binding protein composition may be prepared for storage by mixing the selected composition having the desired degree of purity with optional formulation agents (REMINGTON'S PHARMACEUTICAL SCIENCES, supra) in the form of a lyophilized cake or an aqueous solution. Further, in certain embodiments, the antigen binding protein product may be formulated as a lyophilizate using appropriate excipients such as sucrose.

The pharmaceutical compositions of the invention can be selected for parenteral delivery. Alternatively, the compositions may be selected for inhalation or for delivery through the digestive tract, such as orally. Preparation of such pharmaceutically acceptable compositions is within the skill of the art. The formulation components are present preferably in concentrations that are acceptable to the site of administration. In certain embodiments, buffers are used to maintain the composition at physiological pH or at a slightly lower pH, typically within a pH range of from about 5 to about 8.

When parenteral administration is contemplated, the therapeutic compositions for use in this invention may be provided in the form of a pyrogen-free, parenterally acceptable aqueous solution comprising the desired antigen binding protein in a pharmaceutically acceptable vehicle. A particularly suitable vehicle for parenteral injection is sterile distilled water in which the antigen binding protein is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used, having the effect of promoting sustained duration in the circulation. In certain embodiments, implantable drug delivery devices may be used to introduce the desired antigen binding protein.

Pharmaceutical compositions of the invention can be formulated for inhalation. In these embodiments, antigen binding proteins are advantageously formulated as a dry, inhalable powder. In specific embodiments, antigen binding protein inhalation solutions may also be formulated with a propellant for aerosol delivery. In certain embodiments, solutions may be nebulized. Pulmonary administration and formulation methods therefore are further described in International Patent Application No. PCT/US94/001875, which is incorporated by reference and describes pulmonary delivery of chemically modified proteins.

It is also contemplated that formulations can be administered orally. Antigen binding proteins that are administered in this fashion can be formulated with or without carriers customarily used in the compounding of solid dosage forms such as tablets and capsules. In certain embodiments, a capsule may be designed to release the active portion of the formulation at the point in the gastrointestinal tract when bioavailability is maximized and pre-systemic degradation is minimized. Additional agents can be included to facilitate absorption of the antigen binding protein. Diluents, flavorings, low melting point waxes, vegetable oils, lubricants, suspending agents, tablet disintegrating agents, and binders may also be employed.

Additional pharmaceutical compositions will be evident to those skilled in the art, including formulations involving antigen binding proteins in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, International Patent Application No. PCT/US93/00829, which is incorporated by reference and describes controlled release of porous polymeric microparticles for delivery of pharmaceutical compositions. Sustained-release preparations may include semipermeable polymer matrices in the form of shaped articles, e.g., films, or microcapsules. Sustained release matrices may include polyesters, hydrogels, polylactides (as disclosed in U.S. Pat. No. 3,773,919 and European Patent Application Publication No. EP058481, each of which is incorporated by reference), copolymers of L-glutamic acid and gamma ethyl-L-glutamate (Sidman et al., 1983, Biopolymers 2:547-556), poly (2-hydroxyethyl-methacrylate) (Langer et al., 1981, J. Biomed. Mater. Res. 15:167-277 and Langer, 1982, Chem. Tech. 12:98-105), ethylene vinyl acetate (Langer et al., 1981, supra) or poly-D(−)-3-hydroxybutyric acid (European Patent Application Publication No. EP133988). Sustained release compositions may also include liposomes that can be prepared by any of several methods known in the art. See, e.g., Eppstein et al., 1985, Proc. Natl. Acad. Sci. U.S.A. 82:3688-3692; European Patent Application Publication Nos. EP036676; EP088046 and EP143949, incorporated by reference.

Pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions generally are placed into a container having a sterile access port, for example, an intravenous solution bag or vial having a stopper pierceable by a hypodermic injection needle.

Aspects of the invention includes self-buffering antigen binding protein formulations, which can be used as pharmaceutical compositions, as described in international patent application WO06138181A2 (PCT/US2006/022599), which is incorporated by reference in its entirety herein.

As discussed above, certain embodiments provide antigen binding protein compositions, particularly pharmaceutical antigen binding protein compositions, that comprise, in addition to the antigen binding protein, one or more excipients such as those illustratively described in this section and elsewhere herein. Excipients can be used in the invention in this regard for a wide variety of purposes, such as adjusting physical, chemical, or biological properties of formulations, such as adjustment of viscosity, and or processes of the invention to improve effectiveness and or to stabilize such formulations and processes against degradation and spoilage due to, for instance, stresses that occur during manufacturing, shipping, storage, pre-use preparation, administration, and thereafter.

A variety of expositions are available on protein stabilization and formulation materials and methods useful in this regard, such as Arakawa et al., “Solvent interactions in pharmaceutical formulations,” Pharm Res. 8(3): 285-91 (1991); Kendrick et al., “Physical stabilization of proteins in aqueous solution,” in: RATIONAL DESIGN OF STABLE PROTEIN FORMULATIONS: THEORY AND PRACTICE, Carpenter and Manning, eds. Pharmaceutical Biotechnology. 13: 61-84 (2002), and Randolph et al., “Surfactant-protein interactions,” Pharm Biotechnol. 13: 159-75 (2002), each of which is herein incorporated by reference in its entirety, particularly in parts pertinent to excipients and processes of the same for self-buffering protein formulations in accordance with the current invention, especially as to protein pharmaceutical products and processes for veterinary and/or human medical uses.

Salts may be used in accordance with certain embodiments of the invention to, for example, adjust the ionic strength and/or the isotonicity of a formulation and/or to improve the solubility and/or physical stability of a protein or other ingredient of a composition in accordance with the invention.

As is well known, ions can stabilize the native state of proteins by binding to charged residues on the protein's surface and by shielding charged and polar groups in the protein and reducing the strength of their electrostatic interactions, attractive, and repulsive interactions. Ions also can stabilize the denatured state of a protein by binding to, in particular, the denatured peptide linkages (—CONH) of the protein. Furthermore, ionic interaction with charged and polar groups in a protein also can reduce intermolecular electrostatic interactions and, thereby, prevent or reduce protein aggregation and insolubility.

Ionic species differ significantly in their effects on proteins. A number of categorical rankings of ions and their effects on proteins have been developed that can be used in formulating pharmaceutical compositions in accordance with the invention. One example is the Hofmeister series, which ranks ionic and polar non-ionic solutes by their effect on the conformational stability of proteins in solution. Stabilizing solutes are referred to as “kosmotropic.” Destabilizing solutes are referred to as “chaotropic.” Kosmotropes commonly are used at high concentrations (e.g., >1 molar ammonium sulfate) to precipitate proteins from solution (“salting-out”). Chaotropes commonly are used to denture and/or to solubilize proteins (“salting-in”). The relative effectiveness of ions to “salt-in” and “salt-out” defines their position in the Hofmeister series.

Free amino acids can be used in antigen binding protein formulations in accordance with various embodiments of the invention as bulking agents, stabilizers, and antioxidants, as well as other standard uses. Lysine, proline, serine, and alanine can be used for stabilizing proteins in a formulation. Glycine is useful in lyophilization to ensure correct cake structure and properties. Arginine may be useful to inhibit protein aggregation, in both liquid and lyophilized formulations. Methionine is useful as an antioxidant.

Polyols include sugars, e.g., mannitol, sucrose, and sorbitol and polyhydric alcohols such as, for instance, glycerol and propylene glycol, and, for purposes of discussion herein, polyethylene glycol (PEG) and related substances. Polyols are kosmotropic. They are useful stabilizing agents in both liquid and lyophilized formulations to protect proteins from physical and chemical degradation processes. Polyols also are useful for adjusting the tonicity of formulations.

Among polyols useful in select embodiments of the invention is mannitol, commonly used to ensure structural stability of the cake in lyophilized formulations. It ensures structural stability to the cake. It is generally used with a lyoprotectant, e.g., sucrose. Sorbitol and sucrose are among preferred agents for adjusting tonicity and as stabilizers to protect against freeze-thaw stresses during transport or the preparation of bulks during the manufacturing process. Reducing sugars (which contain free aldehyde or ketone groups), such as glucose and lactose, can glycate surface lysine and arginine residues. Therefore, they generally are not among preferred polyols for use in accordance with the invention. In addition, sugars that form such reactive species, such as sucrose, which is hydrolyzed to fructose and glucose under acidic conditions, and consequently engenders glycation, also is not among preferred polyols of the invention in this regard. PEG is useful to stabilize proteins and as a cryoprotectant and can be used in the invention in this regard.

Embodiments of the antigen binding protein formulations further comprise surfactants. Protein molecules may be susceptible to adsorption on surfaces and to denaturation and consequent aggregation at air-liquid, solid-liquid, and liquid-liquid interfaces. These effects generally scale inversely with protein concentration. These deleterious interactions generally scale inversely with protein concentration and typically are exacerbated by physical agitation, such as that generated during the shipping and handling of a product.

Surfactants routinely are used to prevent, minimize, or reduce surface adsorption. Useful surfactants in the invention in this regard include polysorbate 20, polysorbate 80, other fatty acid esters of sorbitan polyethoxylates, and poloxamer 188.

Surfactants also are commonly used to control protein conformational stability. The use of surfactants in this regard is protein-specific since, any given surfactant typically will stabilize some proteins and destabilize others.

Polysorbates are susceptible to oxidative degradation and often, as supplied, contain sufficient quantities of peroxides to cause oxidation of protein residue side-chains, especially methionine. Consequently, polysorbates should be used carefully, and when used, should be employed at their lowest effective concentration. In this regard, polysorbates exemplify the general rule that excipients should be used in their lowest effective concentrations.

Embodiments of antigen binding protein formulations further comprise one or more antioxidants. To some extent deleterious oxidation of proteins can be prevented in pharmaceutical formulations by maintaining proper levels of ambient oxygen and temperature and by avoiding exposure to light. Antioxidant excipients can be used as well to prevent oxidative degradation of proteins. Among useful antioxidants in this regard are reducing agents, oxygen/free-radical scavengers, and chelating agents. Antioxidants for use in therapeutic protein formulations in accordance with the invention preferably are water-soluble and maintain their activity throughout the shelf life of a product. EDTA is a preferred antioxidant in accordance with the invention in this regard.

Antioxidants can damage proteins. For instance, reducing agents, such as glutathione in particular, can disrupt intramolecular disulfide linkages. Thus, antioxidants for use in the invention are selected to, among other things, eliminate or sufficiently reduce the possibility of themselves damaging proteins in the formulation.

Formulations in accordance with the invention may include metal ions that are protein co-factors and that are necessary to form protein coordination complexes, such as zinc necessary to form certain insulin suspensions. Metal ions also can inhibit some processes that degrade proteins. However, metal ions also catalyze physical and chemical processes that degrade proteins.

Magnesium ions (10-120 mM) can be used to inhibit isomerization of aspartic acid to isoaspartic acid. Ca⁺² ions (up to 100 mM) can increase the stability of human deoxyribonuclease. Mg⁺², Mn⁺², and Zn⁺², however, can destabilize rhDNase. Similarly, Ca⁺² and Sr⁺² can stabilize Factor VIII, it can be destabilized by Mg⁺², Mn⁺² and Zn⁺², Cu⁺² and Fe⁺², and its aggregation can be increased by Al⁺³ ions.

Embodiments of the antigen binding protein formulations further comprise one or more preservatives. Preservatives are necessary when developing multi-dose parenteral formulations that involve more than one extraction from the same container. Their primary function is to inhibit microbial growth and ensure product sterility throughout the shelf-life or term of use of the drug product. Commonly used preservatives include benzyl alcohol, phenol and m-cresol. Although preservatives have a long history of use with small-molecule parenterals, the development of protein formulations that includes preservatives can be challenging. Preservatives almost always have a destabilizing effect (aggregation) on proteins, and this has become a major factor in limiting their use in multi-dose protein formulations. To date, most protein drugs have been formulated for single-use only. However, when multi-dose formulations are possible, they have the added advantage of enabling patient convenience, and increased marketability. A good example is that of human growth hormone (hGH) where the development of preserved formulations has led to commercialization of more convenient, multi-use injection pen presentations. At least four such pen devices containing preserved formulations of hGH are currently available on the market. Norditropin (liquid, Novo Nordisk), Nutropin AQ (liquid, Genentech) & Genotropin (lyophilized—dual chamber cartridge, Pharmacia & Upjohn) contain phenol while Somatrope (Eli Lilly) is formulated with m-cresol.

Several aspects need to be considered during the formulation and development of preserved dosage forms. The effective preservative concentration in the drug product must be optimized. This requires testing a given preservative in the dosage form with concentration ranges that confer anti-microbial effectiveness without compromising protein stability.

As might be expected, development of liquid formulations containing preservatives are more challenging than lyophilized formulations. Freeze-dried products can be lyophilized without the preservative and reconstituted with a preservative containing diluent at the time of use. This shortens the time for which a preservative is in contact with the protein, significantly minimizing the associated stability risks. With liquid formulations, preservative effectiveness and stability should be maintained over the entire product shelf-life (about 18 to 24 months). An important point to note is that preservative effectiveness should be demonstrated in the final formulation containing the active drug and all excipient components.

Antigen binding protein formulations generally will be designed for specific routes and methods of administration, for specific administration dosages and frequencies of administration, for specific treatments of specific diseases, with ranges of bio-availability and persistence, among other things. Formulations thus may be designed in accordance with the invention for delivery by any suitable route, including but not limited to orally, aurally, opthalmically, rectally, and vaginally, and by parenteral routes, including intravenous and intraarterial injection, intramuscular injection, and subcutaneous injection.

Once the pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration. The invention also provides kits for producing a single-dose administration unit. The kits of the invention may each contain both a first container having a dried protein and a second container having an aqueous formulation. In certain embodiments of this invention, kits containing single and multi-chambered pre-filled syringes (e.g., liquid syringes and lyosyringes) are provided.

The therapeutically effective amount of an antigen binding protein-containing pharmaceutical composition to be employed will depend, for example, upon the therapeutic context and objectives. One skilled in the art will appreciate that the appropriate dosage levels for treatment will vary depending, in part, upon the molecule delivered, the indication(s) for which the antigen binding protein is being used, the route of administration, and the size (body weight, body surface or organ size) and/or condition (the age and general health) of the patient. In certain embodiments, the clinician may titer the dosage and modify the route of administration to obtain the optimal therapeutic effect. A typical dosage may range from about 0.1 g/kg to up to about 30 mg/kg or more, depending on the factors mentioned above In specific embodiments, the dosage may range from 1.0 μg/kg up to about 20 mg/kg, optionally from 10 μg/kg up to about 10 mg/kg or from 100 μg/kg up to about 5 mg/kg.

A therapeutic effective amount of an antigen binding protein preferably results in a decrease in severity of disease symptoms, in increase in frequency or duration of disease symptom-free periods or a prevention of impairment or disability due to the disease affliction. For treating tumors, a therapeutically effective amount of an antigen binding protein, preferably inhibits cell growth or tumor growth by at least about 20%, at least about 40%, at least about 50%, at least about 60%, at least about 70%, at least about 80%, or at least about 90% relative to untreated patients. The ability of a antigen binding protein to inhibit tumor growth may be evaluated in an animal model predictive of efficacy in human tumors.

Pharmaceutical compositions may be administered using a medical device. Examples of medical devices for administering pharmaceutical compositions are described in U.S. Pat. Nos. 4,475,196; 4,439,196; 4,447,224; 4,447,233; 4,486,194; 4,487,603; 4,596,556; 4,790,824; 4,941,880; 5,064,413; 5,312,335; 5,312,335; 5,383,851; and 5,399,163, all incorporated by reference herein.

### Methods of Treatment

The antigen binding proteins, e.g., Di-IgG, and pharmaceutical compositions thereof are useful in treating diseases or disorders in a patient. The antigen binding proteins may bind one or more targets related to a disease or disorder. Examples of targets related to various diseases and disorders are described in US20090215992 (incorporated herein by reference) and are provided below.

### Autoimmune and Inflammatory Diseases and Disorders

Many proteins have been implicated in general autoimmune and inflammatory responses, including C5, CCL1 (I-309), CCL11 (eotaxin), CCL13 (mcp-4), CCL15 (MIP-Id), CCL16 (HCC-4), CCL17 (TARC), CCL18 (PARC), CCL19, CCL2 (mcp-1), CCL20 (MIP-3a), CCL21 (MIP-2), CCL23 (MPIF-1), CCL24 (MPIF-2/eotaxin-2), CCL25 (TECK), CCL26, CCL3 (MIP-1a), CCL4 (MIP-1b), CCL5 (RANTES), CCL7 (mcp-3), CCL8 (mcp-2), CXCL1, CXCL10 (IP-10), CXCL11 (I-TAC/IP-9), CXCL12 (SDF1), CXCL13, CXCL14, CXCL2, CXCL3, CXCL5 (ENA-78/LIX), CXCL6 (GCP-2), CXCL9, IL-13, IL-8, CCL13 (mcp-4), CCR1, CCR2, CCR3, CCR4, CCR5, CCR6, CCR7, CCR8, CCR9, CX3CR1, IL8RA, XCR1 (CCXCR1), IFNA2, IL-10, IL-13, IL-17C, IL-1A, IL-1B, IL-1F10, IL-1F5, IL-1F6, IL-1F7, IL-1F8, IL-1F9, IL-22, IL-5, IL-9, LTA, LTB, MIF, SCYE1 (endothelial Monocyte-activating cytokine), SPP1, TNF, TNFSF5, IFNA2, IL-10RA, IL-10RB, IL-13, IL-13RA1, IL-5RA, IL-9, IL-9R, ABCF1, BCL6, C3, C4A, CEBPB, CRP, ICEBERG, IL-1R1, IL-1RN, IL-8RB, LTB4R, TOLLIP, FADD, IRAK1, IRAK2, MYD88, NCK2, TNFAIP3, TRADD, TRAF1, TRAF2, TRAF3, TRAF4, TRAF5, TRAF6, TSLP, ST2, ACVR1, ACVR1B, ACVR2, ACVR2B, ACVRL1, CD28, CD3E, CD3G, CD3Z, CD69, CD70, CD80, CD86, CNR1, CTLA4, CYSLTR1, FCER1A, FCER2, FCGR3A, GPR44, HAVCR2, OPRD1, P2RX7, TLR2, TLR3, TLR4, TLR5, TLR6, TLR7, TLR8, TLR9, TLR10, BLR1, CCL1, CCL2, CCL3, CCL4, CCL5, CCL7, CCL8, CCL11, CCL13, CCL15, CCL16, CCL17, CCL18, CCL19, CCL20, CCL21, CCL22, CCL23, CCL24, CCL25, CCR1, CCR2, CCR3, CCR4, CCR5, CCR6, CCR7, CCR8, CCR9, CX3CL1, CX3CR1, CXCL1, CXCL2, CXCL3, CXCL5, CXCL6, CXCL10, CXCL11, CXCL12, CXCL13, CXCR4, GPR2, SCYE1, SDF2, XCL1, XCL2, XCR1, AMH, AMHR2, BMPR1A, BMPR1B, BMPR2, C19orf10 (IL27w), CER1, CSF1, CSF2, CSF3, DKFZp451J0118, FGF2, GF11, IFNA1, IFNB1, IFNG, IGF1, IL-1A, IL-1B, IL-1R1, IL-1R2, IL-2, IL-2RA, IL-2RB, IL-2RG, IL-3, IL-4, IL-4R, IL-5, IL-5RA, IL-6, IL-6R, IL-6ST, IL-7, IL-8, IL-8RA, IL-8RB, IL-9, IL-9R, IL-10, IL-10RA, IL-10RB, IL-11, IL-11RA, IL-12A, IL-12B, IL-12RB1, IL-12RB2, IL-13, IL-13RA1, IL-13RA2, IL-15, IL-15RA, IL-16, IL-17, IL-17R, IL-18, IL-18R1, IL-19, IL-20, IL-30, IL-21, IL-21R, IL-31, IL-32, IL-33, KITLG, LEP, LTA, LTB, LTB4R, LTB4R2, LTBR, MIF, NPPB, PDGFB, TBX21, TDGF1, TGFA, TGFB1, TGFB1I, TGFB2, TGFB3, TGFBI, TGFBR1, TGFBR2, TGFBR3, TH1L, TL1A, BTLA, TNF, TNFRSF1A, TNFRSF1B, TNFRSF7, TNFRSF8, TNFRSF9, TNFRSF11A, TNFRSF21, TNFSF4, TNFSF5, TNFSF6, TNFSF11, VEGF, ZFPM2, and RNF110 (ZNF144).

### Asthma

Targets believed to be related to asthma and may be targeted by an antigen binding protein described herein include, but are not limited to, IL-4, IL-5, IL-13, IL-25, TNFa, IL-1beta, TARC; MDC; TGF-.beta.; LHR agonist; CL25; SPRR2a; SPRR2b; ADAM8. CSF1 (MCSF), CSF2 (GM-CSF), CSF3 (GCSF), FGF2, IFNA1, IFNB1, IFNG, histamine and histamine receptors, IL1A, IL1B, IL2, IL3, IL-4, IL-5, IL-6, IL-7, IL-8, IL-9, IL-10, IL-11, IL-12A, IL-12B, IL-13, IL-14, IL-15, IL-16, IL-17, IL-18, IL-19, KITLG, PDGFB, IL-2RA, IL-4R, IL-5RA, IL-8RA, IL-8RB, IL-12RB1, IL-12RB2, IL-13RA1, IL-13RA2, IL-18R1, IL-33, ST2, TSLP, CCL1, CCL2, CCL3, CCL4, CCL5, CCL7, CCL8, CCL13, CCL17, CCL18, CCL19, CCL20, CCL22, CCL24, CX3CL1, CXCL1, CXCL2, CXCL3, XCL1, CCR2, CCR3, CCR4, CCR5, CCR6, CCR7, CCR8, CX3CR1, GPR2, XCR1, FOS, GATA3, JAK1, JAK3, STAT6, TBX21, TGFB1, TNF, TNFSF6, YY1, CYSLTR1, FCER1A, FCER2, LTB4R, TB4R2, LTBR, and Chitinase.

### Rheumatoid Arthritis

Rheumatoid arthritis (RA), a systemic disease, is characterized by a chronic inflammatory reaction in the synovium of joints and is associated with degeneration of cartilage and erosion of juxta-articular bone. Many pro-inflammatory cytokines including TNF, chemokines, and growth factors are expressed in diseased joints. Systemic administration of anti-TNF antibody or sTNFR fusion protein to mouse models of RA was shown to be anti-inflammatory and joint protective. Clinical investigations in which the activity of TNF in RA patients was blocked with intravenously administered infliximab (Harriman G, Harper L K, Schaible T F. 1999 Summary of clinical trials in rheumatoid arthritis using infliximab, an anti-TNFalpha treatment. Ann Rheum Dis 58 Suppl 1:161-4), a chimeric anti-TNF monoclonal antibody (mAB), has provided evidence that TNF regulates IL-6, IL-8, MCP-1, and VEGF production, recruitment of immune and inflammatory cells into joints, angiogenesis, and reduction of blood levels of matrix metalloproteinases-1 and -3. A better understanding of the inflammatory pathway in rheumatoid arthritis has led to identification of other therapeutic targets involved in rheumatoid arthritis. Promising treatments such as interleukin-6 antagonists (IL-6 receptor antibody MRA, developed by Chugai, Roche (see Nishimoto, Norihiro et al., Arthritis & Rheumatism (2004), 50(6), 1761-1769), CTLA4Ig (abatacept, Genovese Mc et al 2005 Abatacept for rheumatoid arthritis refractory to tumor necrosis factor alpha inhibition. N Engl J. Med. 353:1114-23.), and anti-B cell therapy (rituximab, Okamoto H, Kamatani N. 2004 Rituximab for rheumatoid arthritis. N Engl J. Med. 351:1909) have already been tested in randomized controlled trials over the past year. Other cytokines have been identified and have been shown to be of benefit in animal models, including interleukin-15 (therapeutic antibody HuMax-IL.sub.-15, AMG 714 see Baslund, Bo et al., Arthritis & Rheumatism (2005), 52(9), 2686-2692), interleukin-17, and interleukin-18, and clinical trials of these agents are currently under way. Dual-specific antibody therapy, combining anti-TNF and another mediator, has great potential in enhancing clinical efficacy and/or patient coverage. For example, blocking both TNF and VEGF can potentially eradicate inflammation and angiogenesis, both of which are involved in pathophysiology of RA. Blocking other pairs of targets involved in RA including, but not limited to, TNF and IL-18; TNF and IL-12; TNF and IL-23; TNF and IL-1beta; TNF and MIF; TNF and IL-17; and TNF and IL-15 with specific Di-IgGs is also contemplated. In addition to routine safety assessments of these target pairs, specific tests for the degree of immunosuppression may be warranted and helpful in selecting the best target pairs (see Luster et al., Toxicology (1994), 92 (1-3), 229-43; Descotes, et al., Developments in biological standardization (1992), 77 99-102; Hart et al., Journal of Allergy and Clinical Immunology (2001), 108(2), 250-257). Whether a Di-IgG molecule will be useful for the treatment of rheumatoid arthritis can be assessed using pre-clinical animal RA models such as the collagen-induced arthritis mouse model. Other useful models are also well known in the art (see Brand D D., Comp Med. (2005) 55(2):114-22). Based on the cross-reactivity of the parental antibodies for human and mouse othologues (e.g. reactivity for human and mouse TNF, human and mouse IL-15 etc.) validation studies in the mouse CIA model may be conducted with “matched surrogate antibody” derived Di-IgG molecules; briefly, a Di-IgG based on two (or more) mouse target specific antibodies may be matched to the extent possible to the characteristics of the parental human or humanized antibodies used for human Di-IgG construction (similar affinity, similar neutralization potency, similar half-life etc.).

### SLE

The immunopathogenic hallmark of SLE is the polyclonal B cell activation, which leads to hyperglobulinemia, autoantibody production and immune complex formation. The fundamental abnormality appears to be the failure of T cells to suppress the forbidden B cell clones due to generalized T cell dysregulation. In addition, B and T-cell interaction is facilitated by several cytokines such as IL-10 as well as co-stimulatory molecules such as CD40 and CD40L, B7 and CD28 and CTLA4, which initiate the second signal. These interactions together with impaired phagocytic clearance of immune complexes and apoptotic material, perpetuate the immune response with resultant tissue injury. The following targets may be involved in SLE and can potentially be used for Di-IgG approach for therapeutic intervention: B cell targeted therapies: CD-20, CD-22, CD-19, CD28, CD4, CD80, HLA-DRA, IL10, IL2, IL-4, TNFRSF5, TNFRSF6, TNFSF5, TNFSF6, BLR1, HDAC4, HDAC5, HDAC7A, HDAC9, ICOSL, IGBP1, MS4A1, RGS1, SLA2, CD81, IFNB1, IL10, TNFRSF5, TNFRSF7, TNFSF5, AICDA, BLNK, GALNAC4S-6ST, HDAC4, HDAC5, HDAC7A, HDAC9, IL10, IL11, IL-4, INHA, INHBA, KLF6, TNFRSF7, CD28, CD38, CD69, CD80, CD83, CD86, DPP4, FCER2, IL2RA, TNFRSF8, TNFSF7, CD24, CD37, CD40, CD72, CD74, CD79A, CD79B, CR2, IL1R2, ITGA2, ITGA3, MS4A1, ST6GAL1, CD1C, CHST10, HLA-A, HLA-DRA, and NT5E; co-stimulatory signals: CTLA4 or B7.1/B7.2; inhibition of B cell survival: BlyS, BAFF; Complement inactivation: C5; Cytokine modulation: the key principle is that the net biologic response in any tissue is the result of a balance between local levels of proinflammatory or anti-inflammatory cytokines (see Sfikakis P P et al 2005 Curr Opin Rheumatol 17:550-7). SLE is considered to be a Th-2 driven disease with documented elevations in serum IL-4, IL-6, IL-10. Di-IgG capable of binding one or more targets selected from the group consisting of IL-4, IL-6, IL-10, IFN-a, and TNF-α are also contemplated. Combination of targets discussed above will enhance therapeutic efficacy for SLE which can be tested in a number of lupus preclinical models (see Peng S L (2004) Methods Mol. Med.; 102:227-72). Based on the cross-reactivity of the parental antibodies for human and mouse othologues (e.g. reactivity for human and mouse CD20, human and mouse Interferon alpha etc.) validation studies in a mouse lupus model may be conducted with “matched surrogate antibody” derived Di-IgG molecules; briefly, a Di-IgG based two (or more) mouse target specific antibodies may be matched to the extent possible to the characteristics of the parental human or humanized antibodies used for human Di-IgG construction (similar affinity, similar neutralization potency, similar half-life etc.).

### Multiple Sclerosis

Multiple sclerosis (MS) is a complex human autoimmune-type disease with a predominantly unknown etiology. Immunologic destruction of myelin basic protein (MBP) throughout the nervous system is the major pathology of multiple sclerosis. MS is a disease of complex pathologies, which involves infiltration by CD4+ and CD8+ T cells and of response within the central nervous system. Expression in the CNS of cytokines, reactive nitrogen species and costimulator molecules have all been described in MS. Of major consideration are immunological mechanisms that contribute to the development of autoimmunity. In particular, antigen expression, cytokine and leukocyte interactions, and regulatory T cells, which help balance/modulate other T cells such as Th1 and Th2 cells, are important areas for therapeutic target identification.

One aspect of the invention pertains to Di-IgG molecules capable of binding one or more, preferably two, targets selected from the group consisting of IL-12, TWEAK, IL-23, CXCL13, CD40, CD40L, IL-18, VEGF, VLA-4, TNF, CD45RB, CD200, IFNgamma, GM-CSF, FGF, C5, CD52, and CCR2. A preferred embodiment includes a dual-specific anti-IL-12/TWEAK Di-IgG as a therapeutic agent beneficial for the treatment of MS.

Several animal models for assessing the usefulness of the Di-IgG molecules to treat MS are known in the art (see Steinman L, et al., (2005) Trends Immunol. 26(11):565-71; Lublin F D., et al., (1985) Springer Semin Immunopathol. 8(3):197-208; Genain C P, et al., (1997) J Mol. Med. 75(3):187-97; Tuohy V K, et al., (1999) J Exp Med. 189(7):1033-42; Owens T, et al., (1995) Neurol Clin. 13(1):51-73; and 't Hart B A, et al., (2005) J Immunol 175(7):4761-8. Based on the cross-reactivity of the parental antibodies for human and animal species othologues (e.g. reactivity for human and mouse IL-12, human and mouse TWEAK etc.) validation studies in the mouse EAE model may be conducted with “matched surrogate antibody” derived Di-IgG molecules; briefly, a Di-IgG based on two (or more) mouse target specific antibodies may be matched to the extent possible to the characteristics of the parental human or humanized antibodies used for human Di-IgG construction (similar affinity, similar neutralization potency, similar half-life etc.). The same concept applies to animal models in other non-rodent species, where a “matched surrogate antibody” derived Di-IgG would be selected for the anticipated pharmacology and possibly safety studies. In addition to routine safety assessments of these target pairs specific tests for the degree of immunosuppression may be warranted and helpful in selecting the best target pairs (see Luster et al., Toxicology (1994), 92 (1-3), 229-43; Descotes, et al., Developments in biological standardization (1992), 77 99-102; Jones R. 2000 Rovelizumab (ICOS Corp). IDrugs. 3(4):442-6).

### Neurological Disorders

Neurodegenerative Diseases

Chronic neurodegenerative diseases are usually age-dependent diseases characterized by progressive loss of neuronal functions (neuronal cell death, demyelination), loss of mobility and loss of memory. Emerging knowledge of the mechanisms underlying chronic neurodegenerative diseases (e g Alzheimer's disease) show a complex etiology and a variety of factors have been recognized to contribute to their development and progression e.g. age, glycemic status, amyloid production and multimerization, accumulation of advanced glycation-end products (AGE) which bind to their receptor RAGE (receptor for AGE), increased brain oxidative stress, decreased cerebral blood flow, neuroinflammation including release of inflammatory cytokines and chemokines, neuronal dysfunction and microglial activation. Thus these chronic neurodegenerative diseases represent a complex interaction between multiple cell types and mediators. Treatment strategies for such diseases are limited and mostly constitute either blocking inflammatory processes with non-specific anti-inflammatory agents (eg corticosteroids, COX inhibitors) or agents to prevent neuron loss and/or synaptic functions. These treatments fail to stop disease progression. Recent studies suggest that more targeted therapies such as antibodies to soluble A-b peptide (including the A-b oligomeric forms) can not only help stop disease progression but may help maintain memory as well. These preliminary observations suggest that specific therapies targeting more than one disease mediator (e.g. A-b and a pro-inflammatory cytokine such as TNF) may provide even better therapeutic efficacy for chronic neurodegenerative diseases than observed with targeting a single disease mechanism (e.g. soluble A-balone) (see C. E. Shepherd, et al, Neurobiol Aging. 2005 Oct. 24; Nelson R B., Curr Pharm Des. 2005; 11:3335; William L. Klein.; Neurochem Int. 2002; 41:345; Michelle C Janelsins, et al., J. Neuroinflammation. 2005; 2:23; Soloman B., Curr Alzheimer Res. 2004; 1:149; Igor Klyubin, et al., Nat. Med. 2005; 11:556-61; Arancio O, et al., EMBO Journal (2004) 1-10; Bornemann K D, et al., Am J. Pathol. 2001; 158:63; Deane R, et al., Nat. Med. 2003; 9:907-13; and Eliezer Masliah, et al., Neuron. 2005; 46:857).

The Di-IgG molecules of the invention can bind one or more targets involved in Chronic neurodegenerative diseases such as Alzheimers. Such targets include, but are not limited to, any mediator, soluble or cell surface, implicated in AD pathogenesis e.g AGE (S100 A, amphoterin), pro-inflammatory cytokines (e.g. IL-1), chemokines (e.g. MCP 1), molecules that inhibit nerve regeneration (e.g. Nogo, RGM A), molecules that enhance neurite growth (neurotrophins) The efficacy of Di-IgG molecules can be validated in pre-clinical animal models such as the transgenic mice that over-express amyloid precursor protein or RAGE and develop Alzheimer's disease-like symptoms. In addition, Di-IgG molecules can be constructed and tested for efficacy in the animal models and the best therapeutic Di-IgG can be selected for testing in human patients. Di-IgG molecules can also be employed for treatment of other neurodegenerative diseases such as Parkinson's disease. Alpha-Synuclein is involved in Parkinson's pathology. A Di-IgG capable of targeting alpha-synuclein and inflammatory mediators such as TNF, IL-1, MCP-1 can prove effective therapy for Parkinson's disease and are contemplated in the invention.

### Neuronal Regeneration and Spinal Cord Injury

Despite an increase in knowledge of the pathologic mechanisms, spinal cord injury (SCI) is still a devastating condition and represents a medical indication characterized by a high medical need. Most spinal cord injuries are contusion or compression injuries and the primary injury is usually followed by secondary injury mechanisms (inflammatory mediators e.g. cytokines and chemokines) that worsen the initial injury and result in significant enlargement of the lesion area, sometimes more than 10-fold. These primary and secondary mechanisms in SCI are very similar to those in brain injury caused by other means e.g. stroke. No satisfying treatment exists and high dose bolus injection of methylprednisolone (MP) is the only used therapy within a narrow time window of 8 h post injury. This treatment, however, is only intended to prevent secondary injury without causing any significant functional recovery. It is heavily criticized for the lack of unequivocal efficacy and severe adverse effects, like immunosuppression with subsequent infections and severe histopathological muscle alterations. No other drugs, biologics or small molecules, stimulating the endogenous regenerative potential are approved, but promising treatment principles and drug candidates have shown efficacy in animal models of SCI in recent years. To a large extent the lack of functional recovery in human SCI is caused by factors inhibiting neurite growth, at lesion sites, in scar tissue, in myelin as well as on injury-associated cells. Such factors are the myelin-associated proteins NogoA, OMgp and MAG, RGM A, the scar-associated CSPG (Chondroitin Sulfate Proteoglycans) and inhibitory factors on reactive astrocytes (some semaphorins and ephrins). However, at the lesion site not only growth inhibitory molecules are found but also neurite growth stimulating factors like neurotrophins, laminin, L1 and others. This ensemble of neurite growth inhibitory and growth promoting molecules may explain that blocking single factors, like NogoA or RGM A, resulted in significant functional recovery in rodent SCI models, because a reduction of the inhibitory influences could shift the balance from growth inhibition to growth promotion. However, recoveries observed with blocking a single neurite outgrowth inhibitory molecule were not complete. To achieve faster and more pronounced recoveries either blocking two neurite outgrowth inhibitory molecules e.g Nogo and RGM A, or blocking an neurite outgrowth inhibitory molecule and enhancing functions of a neurite outgrowth enhancing molecule e.g Nogo and neurotrophins, or blocking a neurite outgrowth inhibitory molecule e.g. Nogo and a pro-inflammatory molecule e.g. TNF, may be desirable (see McGee A W, et al., Trends Neurosci. 2003; 26: 193; Marco Domeniconi, et al., J Neurol Sci. 2005; 233:43; Milan Makwanal, et al., FEBS J. 2005; 272:2628; Barry J. Dickson, Science. 2002; 298: 1959; Felicia Yu Hsuan Teng, et al., J Neurosci Res. 2005; 79:273; Tara Karnezis, et al., Nature Neuroscience 2004; 7, 736; Gang Xu, et al:; J. Neurochem. 2004; 91; 1018).

In one aspect, Di-IgGs capable of binding target pairs such as NgR and RGM A; NogoA and RGM A; MAG and RGM A; OMGp and RGM A; RGM A and RGM B; CSPGs and RGM A; aggrecan, midkine, neurocan, versican, phosphacan, Te38 and TNF-a; A.beta. globulomer-specific antibodies combined with antibodies promoting dendrite & axon sprouting are provided. Dendrite pathology is a very early sign of AD and it is known that NOGO A restricts dendrite growth. One can combine such type of ab with any of the SCI-candidate (myelin-proteins) Ab. Other Di-IgGs targets may include any combination of NgR-p75, NgR-Troy, NgR-Nogo66 (Nogo), NgR-Lingo, Lingo-Troy, Lingo-p75, MAG or Omgp. Additionally, targets may also include any mediator, soluble or cell surface, implicated in inhibition of neurite e.g Nogo, Ompg, MAG, RGM A, semaphorins, ephrins, soluble A-b, pro-inflammatory cytokines (e.g. IL-1), chemokines (e.g. MIP la), molecules that inhibit nerve regeneration. The efficacy of anti-nogo/anti-RGM A or similar Di-IgG molecules can be validated in pre-clinical animal models of spinal cord injury. In addition, these Di-IgG molecules can be constructed and tested for efficacy in the animal models and the best therapeutic Di-IgG can be selected for testing in human patients. In addition, Di-IgG molecules can be constructed that target two distinct ligand binding sites on a single receptor e.g. Nogo receptor which binds three ligand Nogo, Ompg, and MAG and RAGE that binds A-b and S100A. Furthermore, neurite outgrowth inhibitors e.g. nogo and nogo receptor, also play a role in preventing nerve regeneration in immunological diseases like multiple sclerosis. Inhibition of nogo-nogo receptor interaction has been shown to enhance recovery in animal models of multiple sclerosis. Therefore, Di-IgG molecules that can block the function of one immune mediator eg a cytokine like IL-12 and a neurite outgrowth inhibitor molecule eg nogo or RGM may offer faster and greater efficacy than blocking either an immune or an neurite outgrowth inhibitor molecule alone.

### Oncological Disorders

Monoclonal antibody therapy has emerged as an important therapeutic modality for cancer (von Mehren M, et al 2003 Monoclonal antibody therapy for cancer Annu Rev Med.; 54:343-69). Antibodies may exert antitumor effects by inducing apoptosis, redirected cytotoxicity, interfering with ligand-receptor interactions, or preventing the expression of proteins that are critical to the neoplastic phenotype. In addition, antibodies can target components of the tumor microenvironment, perturbing vital structures such as the formation of tumor-associated vasculature. Antibodies can also target receptors whose ligands are growth factors, such as the epidermal growth factor receptor. The antibody thus inhibits natural ligands that stimulate cell growth from binding to targeted tumor cells. Alternatively, antibodies may induce an anti-idiotype network, complement-mediated cytotoxicity, or antibody-dependent cellular-cytotoxicity (ADCC). The use of dual-specific antibody that targets two separate tumor mediators will likely give additional benefit compared to a mono-specific therapy. Di-IgGs capable of binding the following pairs of targets to treat oncological disease are also contemplated: IGF1 and IGF2; IGF1/2 and Erb2B; VEGFR and EGFR; CD20 and CD3, CD138 and CD20, CD38 and CD20, CD38 & CD138, CD40 and CD20, CD138 and CD40, CD38 and CD40. Other target combinations include one or more members of the EGF/erb-2/erb-3 family. Other targets (one or more) involved in oncological diseases that Di-IgGs may bind include, but are not limited to those selected from the group consisting of: CD52, CD20, CD19, CD3, CD4, CD8, BMP6, IL12A, IL1A, IL1B, IL2, IL24, INHA, TNF, TNFSF10, BMP6, EGF, FGF1, FGF10, FGF11, FGF12, FGF13, FGF14, FGF16, FGF17, FGF18, FGF19, FGF2, FGF20, FGF21, FGF22, FGF23, FGF3, FGF4, FGF5, FGF6, FGF7, FGF8, FGF9, GRP, IGF1, IGF2, IL12A, IL1A, LIB, IL2, INHA, TGFA, TGFB1, TGFB2, TGFB3, VEGF, CDK2, EGF, FGF10, FGF18, FGF2, FGF4, FGF7, IGF1, IGF1R, IL2, VEGF, BCL2, CD164, CDKN1A, CDKN1B, CDKN1C, CDKN2A, CDKN2B, CDKN2C, CDKN3, GNRH1, IGFBP6, IL1A, IL1B, ODZ1, PAWR, PLG, TGFB1I1, AR, BRCA1, CDK3, CDK4, CDK5, CDK6, CDK7, CDK9, E2F1, EGFR, ENO1, ERBB2, ESR1, ESR2, IGFBP3, IGFBP6, IL2, INSL4, MYC, NOX5, NR6A1, PAP, PCNA, PRKCQ, PRKD1, PRL, TP53, FGF22, FGF23, FGF9, IGFBP3, IL2, INHA, KLK6, TP53, CHGB, GNRH1, IGF1, IGF2, INHA, INSL3, INSL4, PRL, KLK6, SHBG, NR1D1, NR1H3, NR1I3, NR2F6, NR4A3, ESR1, ESR2, NR0B1, NR0B2, NR1D2, NR1H2, NR1H4, NR1I2, NR2C1, NR2C2, NR2E1, NR2E3, NR2F1, NR2F2, NR3C1, NR3C2, NR4A1, NR4A2, NR5A1, NR5A2, NR6A1, PGR, RARB, FGF1, FGF2, FGF6, KLK3, KRT1, APOC1, BRCA1, CHGA, CHGB, CLU, COL1A1, COL6A1, EGF, ERBB2, ERK8, FGF1, FGF10, FGF11, FGF13, FGF14, FGF16, FGF17, FGF18, FGF2, FGF20, FGF21, FGF22, FGF23, FGF3, FGF4, FGF5, FGF6, FGF7, FGF8, FGF9, GNRH1, IGF1, IGF2, IGFBP3, IGFBP6, IL12A, IL1A, L11B, IL2, IL24, INHA, INSL3, INSL4, KLK10, KLK12, KLK13, KLK14, KLK15, KLK3, KLK4, KLK5, KLK6, KLK9, MMP2, MMP9, MSMB, NTN4, ODZ1, PAP, PLAU, PRL, PSAP, SERPINA3, SHBG, TGFA, TIMP3, CD44, CDH1, CDH10, CDH19, CDH20, CDH7, CDH9, CDH1, CDH10, CDH13, CDH18, CDH19, CDH20, CDH7, CDH8, CDH9, ROBO2, CD44, ELK, ITGA1, APC, CD164, COL6A1, MTSS1, PAP, TGFB1I1, AGR2, AIG1, AKAP1, AKAP2, CANT1, CAV1, CDH12, CLDN3, CLN3, CYB5, CYC1, DAB21P, DES, DNCL1, ELAC2, ENO2, ENO3, FASN, FLJ12584, FLJ25530, GAGEB1, GAGEC1, GGT1, GSTP1, HIP1, HUMCYT2A, IL29, K.sub.6HF, KAI1, KRT2A, MIB1, PART1, PATE, PCA3, PIAS2, PIK3CG, PPID, PR1, PSCA, SLC2A2, SLC33A1, SLC43A1, STEAP, STEAP2, TPM1, TPM2, TRPC6, ANGPT1, ANGPT2, ANPEP, ECGF1, EREG, FGF1, FGF2, FIGF, FLT1, JAG1, KDR, LAMAS, NRP1, NRP2, PGF, PLXDC1, STAB 1, VEGF, VEGFC, ANGPTL3, BAI1, COL4A3, IL8, LAMA5, NRP1, NRP2, STAB 1, ANGPTL4, PECAM1, PF4, PROK2, SERPINF1, TNFAIP2, CCL11, CCL2, CXCL1, CXCL10, CXCL3, CXCL5, CXCL6, CXCL9, IFNA1, IFNB1, IFNG, IL1B, IL6, MDK, EDG1, EFNA1, EFNA3, EFNB2, EGF, EPHB4, FGFR3, HGF, IGF1, ITGB3, PDGFA, TEK, TGFA, TGFB1, TGFB2, TGFBR1, CCL2, CDH5, COL18A1, EDG1, ENG, ITGAV, ITGB3, THBS1, THBS2, BAD, BAG1, BCL2, CCNA1, CCNA2, CCND1, CCNE1, CCNE2, CDH1 (E-cadherin), CDKN1B (p27Kip1), CDKN2A (p1161NK4a), COL6A1, CTNNB1 (b-catenin), CTSB (cathepsin B), ERBB2 (Her-2), ESR1, ESR2, F3 (TF), FOSL1 (FRA-1), GATA3, GSN (Gelsolin), IGFBP2, IL2RA, IL6, IL6R, IL6ST (glycoprotein 130), ITGA6 (a6 integrin), JUN, KLK5, KRT19, MAP2K.sub.7 (c-Jun), MKI67 (Ki-67), NGFB (NGF), NGFR, NME1 (NM23A), PGR, PLAU (uPA), PTEN, SERPINB5 (maspin), SERPINE1 (PAI-1), TGFA, THBS1 (thrombospondin-1), TIE (Tie-1), TNFRSF6 (Fas), TNFSF6 (FasL), TOP2A (topoisomerase Iia), TP53, AZGP1 (zinc-a-glycoprotein), BPAG1 (plectin), CDKN1A (p21Wap1/Cip1), CLDN7 (claudin-7), CLU (clusterin), ERBB2 (Her-2), FGF1, FLRT1 (fibronectin), GABRP (GABAa), GNAS1, ID2, ITGA6 (a6 integrin), ITGB4 (b 4 integrin), KLF5 (GC Box BP), KRT19 (Keratin 19), KRTHB6 (hair-specific type II keratin), MACMARCKS, MT3 (metallothionectin-III), MUC1 (mucin), PTGS2 (COX-2), RAC2 (p21Rac2), S100A2, SCGB1D2 (lipophilin B), SCGB2A1 (mammaglobin 2), SCGB2A2 (mammaglobin 1), SPRR1B (Spr1), THBS1, THBS2, THBS4, and TNFAIP2 (B94).

### EXAMPLES

The following non-limiting Examples demonstrate proof-of-concept that a Di-IgG antibody is functional and can possess characteristics amenable to large-scale manufacturing.

### Example 1

This example describes the design and production of a functional bispecific antibody (Di-IgG) having anti-CD40 and anti-IL-12 specificities. The heavy chain of the Di-IgG was derived from an insertion of the VH domain of an anti-murine CD40 antibody, along with a flexible linker having the amino acid sequence GluArgLysGlyGlyGlySerGly at its N-terminus, into the heavy chain of an anti-IL-12 IgG1 antibody at the site between the Cys220 and Asp 221 (Kabat numbering scheme). The light chain of the Di-IgG was derived from fusing the VL domain of the murine CD40 antibody, along with a flexible linker having the amino acid sequence GluArgLysGlyGlyGlySerGly at its N-terminus, to the C-terminus of the light chain of the anti-IL-12 antibody (FIG. 1).

The heavy chain and light chain of the Di-IgG were transiently cotransfected into 293E cells and the resultant Di-IgG antibody was secreted into the culture medium. The Di-IgG was purified from the medium using Protein A chromatography.

The Di-IgG antibody was expressed at very high yield (>100 ug/ml of cell culture). The Di-IgG had the expected molecular size as a fully assembled IgG and consisted of heavy- and light-chains with expected sizes (as determined by Western blotting. Moreover, greater than 85% of the Protein A purified Di-IgG was free of aggregation.

### Example 2

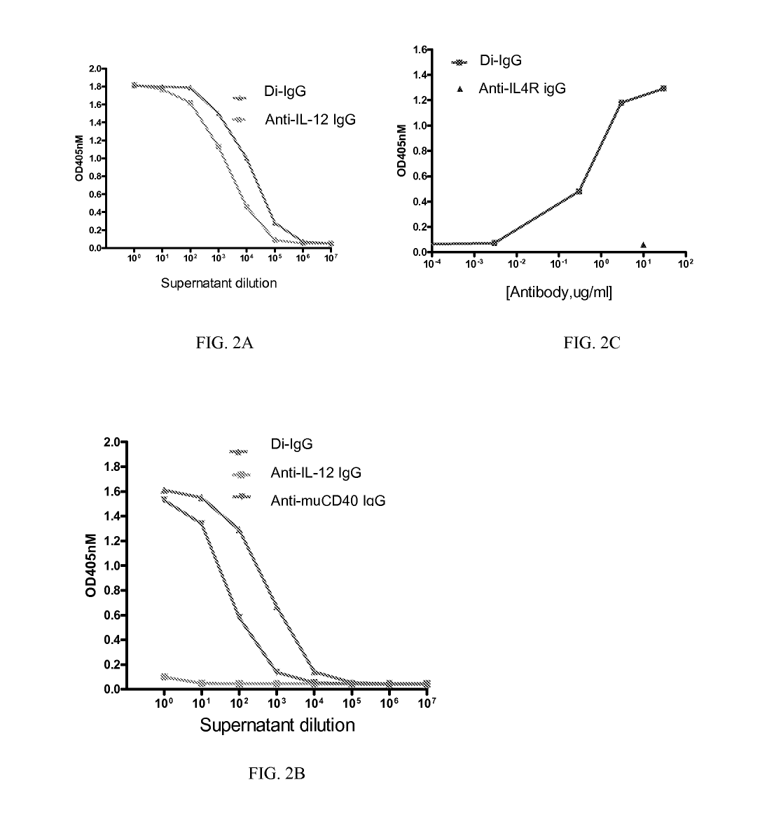

This Example demonstrates that the Di-IgG can bind both targets simultaneously. The Di-IgG created in Example 1 was first tested for binding to human IL-12. Human IL-12 was coated on an ELISA plate. The Di-IgG of Example 1 or the parental IL-12 antibody were added to the well. Bound antibody was detected with an anti-Fc horseradish peroxidase-conjugated secondary antibody. The results are shown in FIG. 2A.

The Di-IgG of Example 1 was then tested for binding to murine CD40 (MuCD40). MuCD40-Fc was coated on an ELISA plate. The Di-IgG of Example 1, the parental IL-12 antibody, or the parental muCD40 antibody were added to the well. Bound antibody was detected with an anti-kappa light chain horseradish peroxidase-conjugated secondary antibody. The results are shown in FIG. 2B.

The Di-IgG of Example 1 failed to bind human 4-1BBFc, human EPO receptor, or human IL-4 receptor, suggesting the Di-IgG is specific to human IL-12 and muCD40.

To demonstrate that the Di-IgG of Example 1 can bind both targets simultaneously, the ELISA technique above was slightly modified. Human IL-12 was coated on the ELISA plate, The Di-IgG or a control IL-4 receptor antibody was then added to the well. Biotinylated muCD40Fc was then added to the well at 2 ug/ml. Presence of the biotinylated meCD40Fc was then detected using streptavidin-conjugated horseradish peroxidase. As shown in FIG. 2C, the Di-IgG of Example 1 was able to bind to IL-12 on the plate and muCD40 in solution at the same time, whereas the control antibody failed to provide any signal in the assay.

### Example 3

Alt et al. FEBS Letters 454 (1999) 90-94 suggests that adding functional variable domains to antibodies can affect the ability of the antibody to bind to Fc receptors. In Alt et al., an scFv was added to the C-terminus of the CH3 domain. The authors state that the constructs were unable to carry out complement-mediated haemolysis and showed a reduced affinity for the Fcγ receptor. These characteristics are often important for the therapeutic value of a given antibody. Because the variable domains in the Di-IgG are situated close to the CH2 domain, which is portion of the antibody most involved in Fc-receptor binding, it was possible the Di-IgG would lose the ability to bind the various Fc receptors.

The FcγR binding activity of Di-IgG and traditional antibodies was compared using AlphaScreening. The FcγRs tested were RI, RIIA, RIIB, and RIIIA(Val158). The Di-IgG retained the binding activity to FcγRs. In the case of FcγRIIIA(Val158), the binding of Di-IgG was stronger than the traditional IgG. Thus, despite suggestions in the art that the addition of variable domains to an antibody at positions other than the end of the “arms” affects the FcγR-binding ability, surprisingly, the Di-IgG do not show evidence of this effect.

### Example 4

Another important characteristic of a therapeutic antibody is thermostability. The thermostability of several various Di-IgG constructs were tested. Surprisingly, despite the fact that a large domain was inserted into the backbone of the heavy chain and added to the C-terminus of the light chain, the Di-IgG constructs exhibited sharp thermal unfolding transitions.

1. An antigen binding protein comprising a first polypeptide chain and a second polypeptide chain, wherein said first polypeptide chain comprises VH1-CH1-X-VH2, wherein:
VH1 is a first heavy chain variable domain;
CH1 is a heavy chain constant domain;
X is an optional linker;
VH2 is a second heavy chain variable domain; and
wherein said second polypeptide chain comprises VL1-CL-X-VL2, wherein:
VL1 is a first light chain variable domain;
CL is a light chain constant domain;
X is an optional linker;
VL2 is a second light chain variable domain.

2. The antigen binding protein of claim 1, wherein the first polypeptide chain comprises VH1-CH1-X-VH2-Fc.

3. The antigen binding protein of claim 2, wherein the Fc is a native Fc.

4. The antigen binding protein of claim 2, wherein the Fc is a variant Fc.

5. The antigen binding protein of claim 2, wherein the Fc is selected from the group consisting of an Fc region from an IgA, IgD, IgE, IgG1, IgG2, IgG3, IgG4, and IgM.

6. The antigen binding protein of claim 1, wherein the CL is a kappa light chain constant domain.

7. The antigen binding protein of claim 1, wherein the CL is a lambda light chain constant domain.

8. The antigen binding protein of claim 1, wherein the first polypeptide chain comprises VH1-CH1-linker-VH2.

9. The antigen binding protein of claim 8, wherein the linker comprises 3 to 50 amino acids.

10. The antigen binding protein of claim 9, wherein the linker comprises a plurality of glycines.

11. The antigen binding protein of claim 10, wherein said linker comprises the amino acid sequence GluArgLysGlyGlyGlySerGly (SEQ ID NO:1).

12. The antigen binding protein of claim 1, wherein the first polypeptide chain comprises VL1-CL-linker-VL2.

13. The antigen binding protein of claim 12, wherein the linker comprises 3 to 50 amino acids.

14. The antigen binding protein of claim 13, wherein said linker comprises a plurality of glycines.

15. The antigen binding protein of claim 14, wherein said linker comprises the amino acid sequence GluArgLysGlyGlyGlySerGly (SEQ ID NO:1).

16. The antigen binding protein of claim 1 comprising two first polypeptide chains and two second polypeptide chains.

17-23. (canceled)

24. The antigen binding protein of claim 1, wherein the VH1 and VL1 specifically bind a first antigen and the VH2 and VL2 specifically bind a second antigen.

25. The antigen binding protein of claim 24, wherein the first antigen and the second antigen are different antigens.

26. The antigen binding protein of claim 24, wherein the first antigen and the second antigen are the same antigen.

27. The antigen binding protein of claim 26, wherein the VH1 and VL1 specifically bind a first epitope on the antigen and the VH2 and VL2 specifically bind a second epitope on the antigen.

28. A nucleic acid encoding a first polypeptide chain comprising VH1-CH1-X-VH2, wherein:
VH1 is a first heavy chain variable domain;
CH1 is a heavy chain constant domain;
X is an optional linker;
VH2 is a second heavy chain variable domain.

29. A nucleic acid encoding a second polypeptide chain comprising VL1-CL-X-VL2, wherein:
VL1 is a first light chain variable domain;
CL is a light chain constant domain;
X is an optional linker;
VL2 is a second light chain variable domain.

30. A host cell comprising the nucleic acid of claim 28.

31. The host cell of claim 30 further comprising a nucleic acid encoding a second polypeptide chain comprising VL1-CL-X-VL2, wherein:
VL1 is a first light chain variable domain;
CL is a light chain constant domain;
X is an optional linker;
VL2 is a second light chain variable domain.

32. A method of making an antigen binding protein, the method comprising:
a) culturing a host cell of claim 32 31 in a culture medium under conditions such that the first polypeptide and second polypeptide are expressed to create a culture; and
b) isolating the antigen binding protein from the culture.

33. The method of making an antigen binding protein of claim 32, wherein the antigen binding protein is isolated from the culture medium.

34. A pharmaceutical composition comprising an antigen binding protein of claim 1 and a pharmaceutically acceptable excipient.
